(19) United States
(12) Patent Application Publication
(10) Pub. No.: US 2009/0318833 A1
(43) Pub. Date: Dec. 24, 2009

(54) Needle Structures and Methods for Fabricating Needle Structures

(75) Inventor: Chee Yen Lim, Singapore (SG)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: AGENCY FOR SCIENCE TECHNOLOGY AND RESEARCH, SINGAPORE (SG)

(21) Appl. No.: 12/441,508

(22) PCT Filed: Sep. 18, 2006
(86) PCT No.: PCT/SG06/00273

§ 371 Date: Aug. 20, 2009

(51) Int. Cl. A61M 5/32; A61B 5/15; A61M 5/34; B23P 11/00; B29C 33/42

(52) U.S. Cl. 600/573; 604/272; 604/239; 29/428; 264/220

(57) ABSTRACT

A needle comprising: a base portion; a stem portion extending from the base portion; a tip portion formed on the stem portion; wherein a length of the stem portion is chosen such that the needle can be used for intradermal or subcutaneous drug delivery, and the base portion, the stem portion and the tip portion are integrally formed.

### FIELD OF INVENTION

The present invention relates broadly to needle structures and methods for fabricating needle structures.

### BACKGROUND

Needle structures such as needles and microneedles are being utilised in various technology fields, for example, in the administration of drugs to a body. Drugs can be administered to the body by, for example, injection. A typical injection operation involves a syringe with a needle which is first used to breach the skin and then to be inserted to reach a desired depth in the body before the drug is injected into the body. There are various modes of delivery of drugs into the body, for example, transdermal delivery, intradermal delivery, subcutaneous delivery, intramuscular delivery, intravenous delivery, etc., which deliver drugs to different parts of the body at different depths. Generally, needle structures should be sharp and robust enough to pierce the skin for the administration of drugs into the body.

The use of conventional microneedles is generally limited to the transdermal mode of delivery of drugs into the body, where the microneedles are inserted through the stratum corneum (SC) and the epidermis layer to deliver drugs shallowly into the skin.

Conventional needles are used for delivery modes such as intradermal delivery, subcutaneous delivery, intramuscular deliver and intravenous delivery where the needles are inserted into greater depths of the body beyond the stratum corneum and epidermis layer of the skin. For example, in the intradermal delivery mode, the needle is inserted within the dermis layer of the skin (beyond the epidermis layer). In subcutaneous delivery, the needle is inserted beyond the thickness of the skin. Intramuscular delivery and intravenous delivery involves inserting the needle into the muscles and veins, respectively, and can generally be classified as a form of subcutaneous delivery. However, these traditional needles are made of metal and are complicated and costly to manufacture. The traditional metal needles are usually fabricated separately and an assembly step is required to assemble the needle with another part, e.g. a luer connector in order for the needle to be attached to a syringe and the needle may require post fabrication machining. Further, customisation of metal needles with complex shapes or geometries can be very difficult to achieve, if not impossible. Further more, fabrication of metal needles is not cost effective due to post fabrication processes such as assembling to the luer connector and customisation of needle geometry.

Therefore, there is a need for alternative needle structures and methods of fabricating the needle structures for intradermal delivery and subcutaneous delivery of drugs into the body.

### SUMMARY

According to a first aspect of the present invention, there is provided a needle comprising: a base portion; a stem portion extending from the base portion; a tip portion formed on the stem portion; wherein a length of the stem portion is chosen such that the needle can be used for intradermal or subcutaneous drug delivery, and the base portion, the stem portion and the tip portion are integrally formed.

The base portion, the stem portion and the tip portion may be integrally formed from the same material.

At least one of the base portion, the stem portion or the tip portion may be integrally formed from a different material.

The needle may further comprise: at least one side port; and

a lumen extending within the needle, the lumen extending from the base portion to the stem portion and the tip portion, wherein the side port extends into the lumen such that the side port and the lumen are in fluid communication with each other.

The lumen may comprise a plurality of sections, each section having a different diameter or width.

The diameter or widths of the plurality of sections may be in descending order starting from a base section of the lumen.

The lumen may be tapered.

The needle may comprise two or more side ports disposed on the tip portion of the needle.

The needle may further comprise an extended tip portion formed on the tip portion, the extended tip portion forming an apex of the needle, wherein the extended tip portion comprises a substantially elongate protrusion extending from the tip portion.

A geometry of the extended tip portion may be substantially the same or different from a geometry of the tip portion.

The base portion may comprise a connection adapter for coupling to a dispensing apparatus.

The base portion may comprise a luer lock structure for interconnecting the needle to a syringe.

The base portion may comprise a barrel portion, the barrel portion being adapted for coupling to a plunger.

The needle may be made from at least one of a group of polymers consisting of polycarbonate (PC), polystyrene (PS), polyetherimide (PEI), polyetherehterketone (PEEK).

The needle may be fabricated using injection moulding or cast moulding or derivatives of cast moulding or injection moulding.

According to a second aspect of the present invention, there is provided a method of fabricating a needle, the method comprising: forming a base portion; forming a stem portion extending from the base portion; forming a tip portion formed on the stem portion; wherein a length of the stem portion is chosen such that the needle can be used for intradermal or subcutaneous drug delivery, and the base portion, the stem portion and the tip portion are integrally formed.

The base portion, the stem portion and the tip portion may be integrally formed from the same material.

At least one of the base portion, the stem portion or the tip portion may be integrally formed from a different material.

The method may further comprise: forming at least one side port; and

forming a lumen extending within the needle, the lumen extending from the base portion to the stem portion and the tip portion, wherein the side port extends into the lumen such that the side port and the lumen are in fluid communication with each other.

The lumen may comprise a plurality of sections, each section having a different diameter or width.

The diameter or widths of the plurality of sections may be in descending order starting from a base section of the lumen.

The lumen may be tapered.

The method may further comprise: two or more side ports disposed on the tip portion of the needle.

The method may further comprise: forming an extended tip portion on the tip portion, wherein the extended tip portion forms an apex of the needle and the extended tip portion comprises a substantially elongate protrusion extending from the tip portion.

The base portion may comprise a connection adapter for coupling to a dispensing apparatus.

The base portion may comprise a luer lock structure for interconnecting the needle to a syringe.

The base portion may comprise a barrel portion, the barrel portion being adapted for coupling to a plunger.

The method may further comprise: forming a master mould comprising a positive shape of the needle; using the master mould to form a production mould comprising a cavity comprising a periphery of a negative shape of the needle; and using the production mould to form the needle.

The method may comprise: forming the master mould comprising a positive shape of the needle; using the master mould to form an intermediate mould comprising a cavity comprising a periphery of a negative shape of the needle; and using the intermediate mould to form the production mould.

The method may comprise: forming a pair of production mould halves, each production mould halve comprising a cavity comprising a periphery of a negative shape of the needle; using the pair of production mould halves to form the needle.

The method may comprise: forming a pair of intermediate mould halves, each intermediate mould halve comprising a cavity comprising a periphery of a negative shape of the needle; using the pair of intermediate mould halves to form a pair of production mould halves.

The pair of production mould halves may be identical to each other, each production mould halve comprising a plurality of cavities, each cavity comprising a periphery of a negative shape of the needle.

The periphery of the negative shape of the needle may be substantially parallel to an interface of the two production mould halves.

The intermediate mould halves may be made from polymer.

The method may further comprise: filling the cavity of the production mould halves with a polymer to form the needle.

The positive shape of the needle on the master mould may be created by precision wire-cutting.

The production mould halves may be made of metal.

The method may further comprise: providing an insert member;

providing the pair of production mould halves, each of the production mould halves further comprising at least one slot portion for receiving and aligning the insert member such that a leading edge of the insert intersects the periphery of the negative shape of the needle; aligning the pair of production mould halves; inserting the insert member into the production mould halves; and filling the production mould halves with a fill material, wherein the insert creates the lumen in the needle and the intersection of the leading edge of the insert with the periphery of the negative shape of the needle creates the side port extending into the lumen such that the lumen and the side port are in fluid communication with each other.

The periphery of the negative shape of the needle may comprise a channel extending from an apex of the negative shape of the needle for forming the extended tip portion of the needle.

The method may further comprise filling the channel at least partially to form the extended tip portion.

The insert member may comprise a pin having a plurality of sections, each section having a different diameter or width.

The pin may be tapered.

According to a third aspect of the present invention, there is provided a use of a needle for injecting liquid into a body.

According to a fourth aspect of the present invention, there is provided a use of a needle for extracting body fluid from a body.

Extracting body fluid from the body may include whole blood sampling.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1(a) is a schematic isometric view of a needle;

FIG. 1(b) is a schematic drawing of a the needle of FIG. 1(a);

FIG. 2 is a schematic drawing of a needle breaching the skin for intradermal or subcutaneous drug delivery;

FIG. 3 is a schematic drawing of another needle breaching the skin for intradermal or subcutaneous drug delivery;

FIG. 4 is a schematic drawing of another needle;

FIGS. 5(a) and 5(b) are schematic drawings of a portion of a needle without an extended tip portion and a needle with an extended tip portion, respectively;

FIG. 6 is schematic drawing of a mould halve 600 used for fabricating a needle;

FIG. 7 is a schematic drawing of a portion of a mould halve used for fabricating a needle;

FIG. 8 is a schematic drawing of a mould halve of another mould halve used for fabricating another needle;

FIGS. 9(a)-9(c) are schematic drawings showing a top view of a process for making a production mould halve for use in fabricating needles;

FIGS. 10(a)-10(c) are schematic drawings showing a top view of another process for making a production mould halve for use in fabricating needles;

FIGS. 11(a)-11(c) are schematic drawings showing a top view of a process for making an intermediate mould halve;

FIGS. 12(a)-12(c) are schematic drawings showing a top view of another process for making an intermediate mould;

FIGS. 13(a) and 13(b) are schematic drawings showing a top view of a process for making a production mould halve using an intermediate mould halve;

FIGS. 14(a) and 14(b) are schematic drawings illustrating an intradermal delivery mode and a subcutaneous delivery mode, respectively; and

FIGS. 15(a)-15(c) are schematic drawings of how skin is compressed and tensioned prior to application of the needle to the skin.

### DETAILED DESCRIPTION

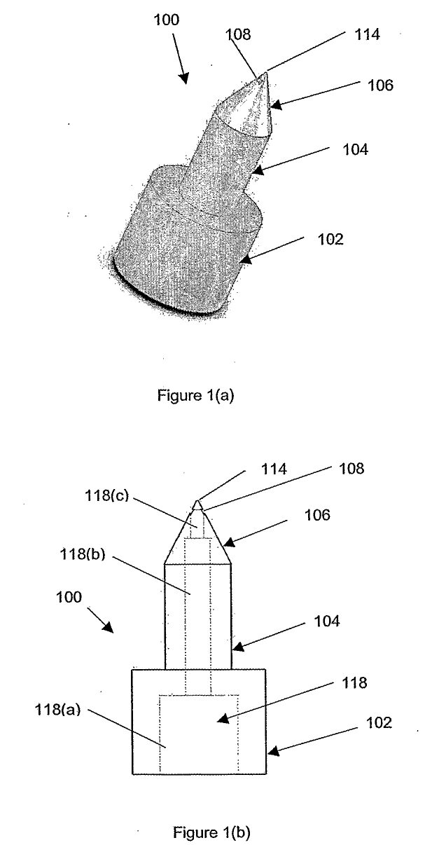

A schematic isometric view of a needle 100 and a schematic drawing of the needle 100 are shown in FIG. 1(a) and FIG. 1(b), respectively. The needle 100 comprises a base portion 102, a stem portion 104 extending from the base portion 102, and a tip portion 106 formed on the stem portion 104. The base portion 102 comprises a connection adapter 102 (e.g. a luer-lock connector) for coupling to a syringe (not shown) such that the needle 100 is interconnected to the syringe. Alternatively, the base portion 102 can comprise a barrel portion adapted for coupling to a plunger.

The tip portion 106 comprises two dispensing ports 108. The two dispensing ports 108 are disposed opposite one another on sides of the tip portion 106 (hence termed side-ports). A lumen structure 118 extends within the needle 100 from the base portion 102 to the stem portion 104 and to the tip portion 106. The lumen structure 118 leads to the two side-ports 108 such that the lumen structure 118 is in fluid connection with the two side-ports 108. The lumen structure 118 terminates at the two side-ports 108 leaving a distance between the side ports 108 and an apex 114 of the needle 100. The lumen structure 118 comprises a plurality of lumen sections 118(a)-(c) with different widths/diameters, as shown in FIG. 1(b). The diameter of the lumen sections 118(a)-(c) are in descending size starting from the lumen section 118(a) of the base portion 102 of the needle 100. The extent to which the width/diameter of the lumen structure 118 intersects with the tip portion 106 of the needle 100 determines the size of the side ports 108.

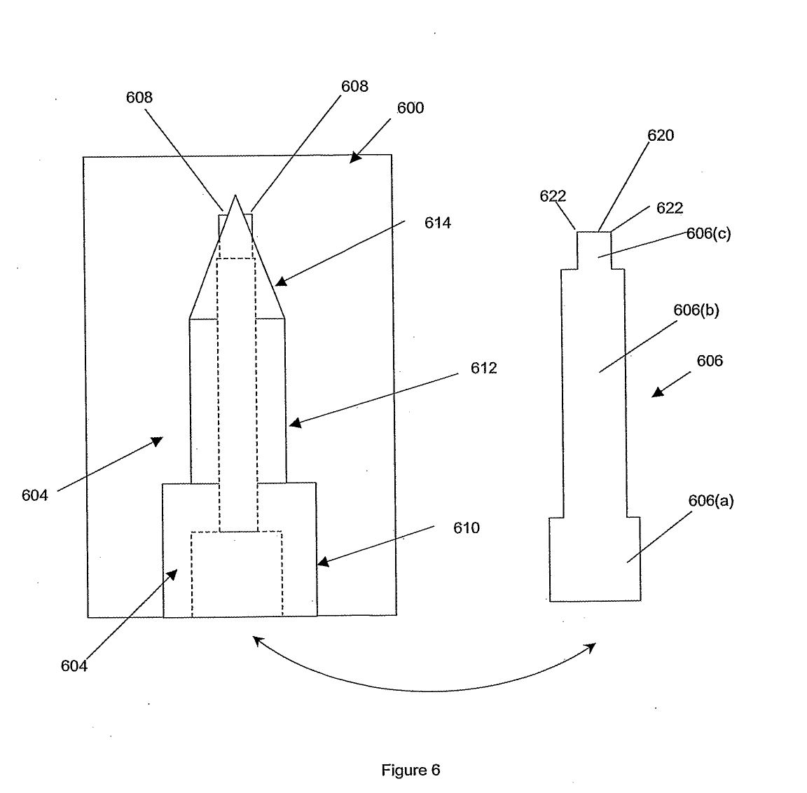

The base portion 102, the stem portion 104 and the tip portion 106 of the needle are formed integrally. Further, the base portion 102, the stem portion 104 and the tip portion 106 are formed from the same material and the entire needle 100 is made from plastic. Alternatively, the base portion 102, the stem portion 104 and the tip portion 106 of the needle 100 can be made using different materials. Methods of fabricating the needle 100 will be described in greater detail with reference to FIGS. 6 to 13.

The needle 100 is approximately between 3 mm to 12.7 mm in length. 50 microns to 1000 microns The lumen section 118(c) can have a width/diameter ranging from about 50 microns to about 200 microns. The lumen section 118(b) can have a width/diameter ranging from about 100 microns to 1000 microns. The lumen section 118(a) can have a width/diameter ranging from about 1 mm to 10 mm. The length of the stem portion 104 is chosen such that the needle 100 can be used for intradermal or subcutaneous drug delivery. It will be appreciated that the length of the stem portion and the lumen structure can have other dimensions or geometries depending on the mode of drug delivery required and design requirements. At least one of the lumen sections 118(a)-118(c) can be tapered, for example, the lumen section 118(c) can have a width/diameter of about 50 microns at one end and a width/diameter of about 200 microns at the other end. Similarly, the lumen sections 118(b) and 118(c) can also be tapered accordingly. It will be appreciated that the lumen structure 118 can be of various shapes, for example, circular or rectangular, etc.

The base portion 102 and the stem portion 104 are cylindrical in shape and the tip portion 106 is generally conical in shape. It will be appreciated that other geometries are possible, for example, the tip portion 106 can be pyramid shape, hexagonal shape, etc.

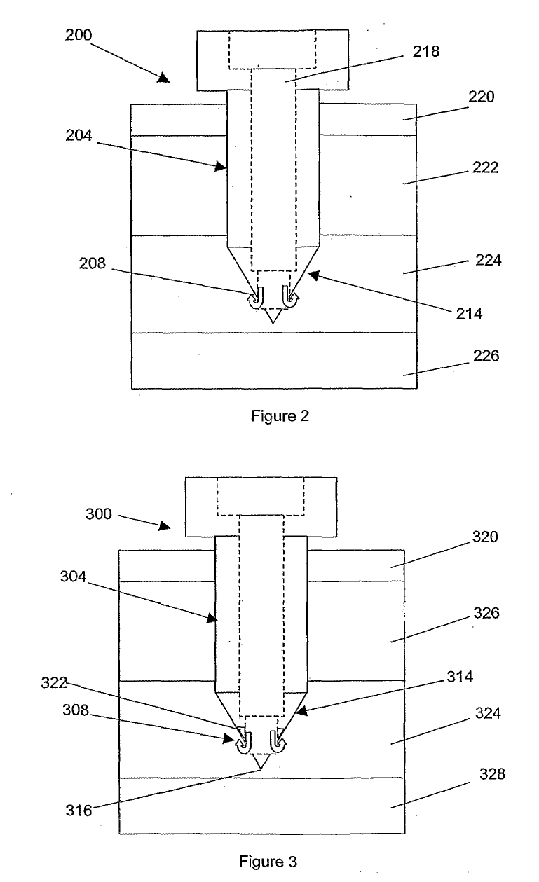

A schematic drawing of a needle 200 breaching the skin 220 for intradermal or subcutaneous drug delivery is shown in FIG. 2. After the stratum corneum 220 of the skin is breached by the needle 200, the needle is inserted further within the skin such that the side ports 208 of the needle reach a desirable depth. For intradermal delivery, the needle is inserted within the dermis layer 224 below the epidermis layer 222 of the skin, as shown in FIG. 2. For subcutaneous delivery (e.g. intramuscular delivery), the needle 200 is inserted beyond the dermis layer 224 and into the muscle layer 226. The drug to be dispensed passes through the lumen 218 and is delivered into the body via the side-ports 208, as indicated by the arrows in FIG. 2.

A schematic drawing of a portion of another needle 300 breaching the skin for intradermal or subcutaneous drug delivery is shown in FIG. 3, each side port 308 of the needle 300 comprises an inwardly chamfered area 322 at a lower portion of the side port 308 towards the stem portion 304 of the needle 300. The inwardly chamfered areas 322 help to dispense drugs more effectively and minimises clogging of the side ports 308 during penetration of the needle 300 into the skin. A portion of the side port 308 together with the apex 318 of the needle 300 serve the purpose of breaching the skin 320 and subsequently expanding and holding the skin in place, so that the drugs can be dispensed through the side ports 308, first into the clearance created by the chamfered areas 322 and then into the body. For intradermal delivery, the needle is inserted within the dermis layer 324 below the epidermis layer 326 of the skin, as shown in FIG. 3. For subcutaneous delivery (e.g. intramuscular delivery), the needle 200 is inserted beyond the dermis layer 324 and into the muscle layer 328.

The distance between the side ports and the apex of the needle, termed the submerged distance, is chosen such that when the needle is used to breach the skin, the distance of the side ports from the apex of the needle is sufficiently far enough for the side ports to be isolated from the breaching and compressing action of the skin yet close enough to the apex of the needle such that the side-ports can be completely buried within the body for effective drug dispensing without spillage. Another consideration is that the nearer the side-ports are to the apex, the weaker the mechanical structure of the needle is. Depending on the materials used, the geometry of the needle and the size and number of the side-ports, the distance between the centre of the side-port from the apex of the needle can vary around 200 microns approximately, for example, between about 100 to 500 microns, for a needle with a tip portion of about 1000 microns in length. The length of the entire needle can range from about 1 mm to 10 mm in length.

In the needles described above, each needle comprises two side-ports. It will be appreciated that at least one side-port (in fluid communication with the lumen) is required to allow dispensation of the drugs and the number of side ports required can be varied depending on design requirements.

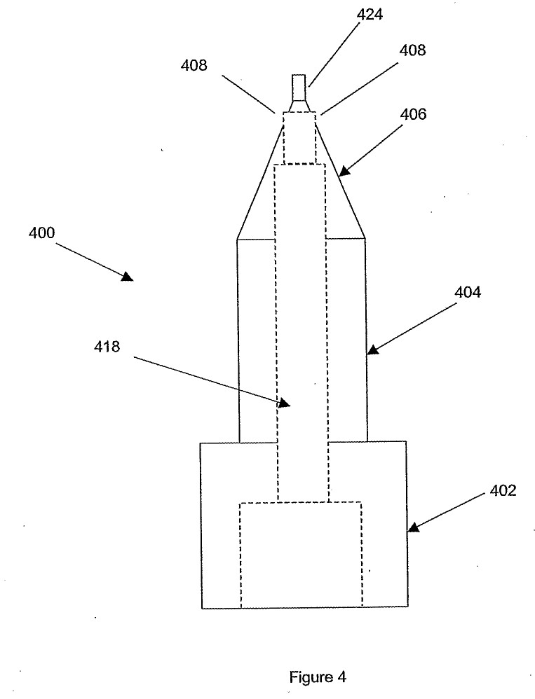

FIG. 4 shows a schematic drawing of another needle 400. In addition to two side-ports 408 and the lumen structure 418, the needle 400 comprises an extended tip portion 424 formed on the tip portion 406. The extended tip portion 424 forms the apex of the needle 400. The extended tip portion 424 comprises a substantially elongate protrusion extending from the tip portion 406 of the needle 400. It will be appreciated that a geometry of the extended tip portion 424 can be substantially the same or different from a geometry of the tip portion 406. The extended tip portion 424 can be of other dimensions, for example, less than 50 μm if required, depending on design requirements.

The needle 400 comprising the base portion 402, the stem portion 404, the tip portion 406 and the extended tip portion 424 are formed integrally. Further, the base portion 402, the stem portion 404, the tip portion 406 and the extended tip portion 424 of the needle 400 are formed from the same material. Alternatively, the base portion 402, the stem portion 404 and the tip portion 406 of the needle 400 can be formed from different materials.

The needles described above can be made from polymers, for example, various grades of polycarbonate (PC), polystyrene (PS), and particularly bio-compatible polymers such as polyetherimide (PEI), polyetherehterketone (PEEK), etc. The type of polymer used to make the needle is chosen based on characteristic mechanical properties of the polymer that are suitable for specific dimensions of the needle designed for specific applications. In addition, each polymer can be filled with additives for the purposes of reinforcement and/or conductivity of the needle.

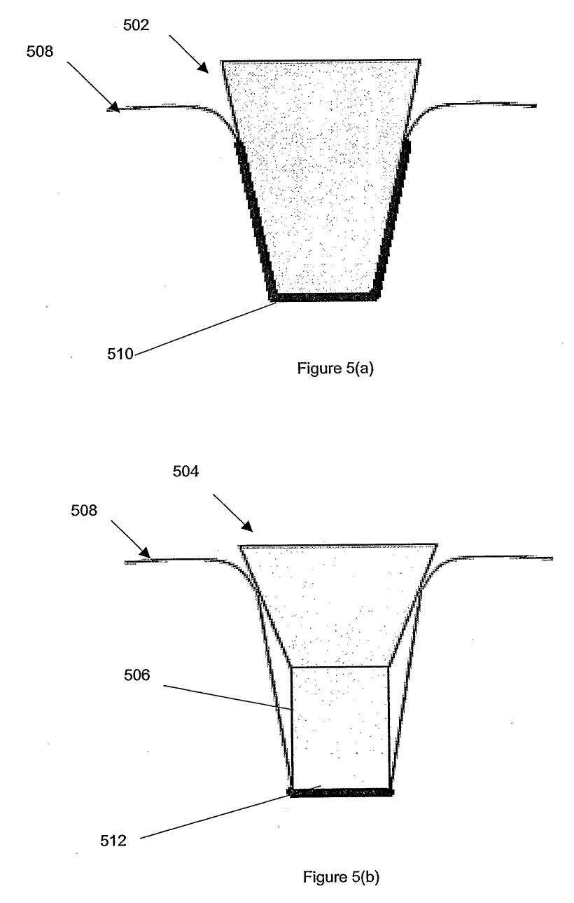

FIGS. 5(a) and 5(b) are schematic drawings of a portion of a needle 502 without an extended tip portion and a needle 504 with an extended tip portion 506, used to breach the stratum corneum (SC) of skin 508 for intradermal or subcutaneous drug delivery. The side ports of the needles 502, 504 are not shown in FIGS. 5(a) and 5(b). The extended tip portion 506 of the needle 504 in FIG. 5(b) has the same width as an apex 510 of the needle 502 without the extended tip portion. Due to the highly deformable nature of skin (which is also viscoelastic), initial contact with the apex 510 of the needle 502 normally deforms and tensions the skin 508 for a signification portion of an advancing stroke of the needle 502 before breach of the skin 508 occurs. During this deformation stage, the skin 508 is forced to conform to the shape of the needle 502, particularly to the geometry of the apex 510. Therefore, comparing the needle 502 without the extended tip and the needle 504 with the extended tip portion 506, the needle 502 with a “normal” tip (i.e. without any extended tip portion) causes full conformance of the skin 508 to the geometry of the tip 510. This in turn results in a larger effective contact area between the skin 508 and the needle, as shown in FIG. 5(a). The effective contact areas are indicated by the bold lines in FIGS. 5(a) and 5(b). Since a threshold pressure must be reached before any puncturing or breach of the SC of the skin 408 can occur, a larger contact area reduces the resultant pressure caused by the apex 510 of the needle 502 on the skin 508 and makes breaching of the SC more difficult as more force has to be applied. However, for the needle 504 with the extended tip portion 506, the effective contact area between the skin and the needle 504 is restricted to the apex 512 of the extended tip portion 506 which has a smaller area compared to the effective contact area in FIG. 5(a). As a result of the reduced effective contact area, a higher pressure is exerted on the skin by the needle 504 and less force is applied to breach the SC of the skin 508.

A schematic drawing of a mould halve 600 used for fabricating a needle is shown in FIG. 6. Two identical mould halves 600 are used to form a mould (not shown), therefore, only one mould halve 600 is shown and described. The mould halve 600 in FIG. 6 can be used to fabricate, for example, the needle 100 in FIGS. 1(a) and 1(b). The mould halve 600 comprises a cavity 604 defining a periphery of a negative shape of the needle to be fabricated, (e.g. needle 100 in FIG. 1(a)) The cavity 604 comprises a base portion 610, a stem portion 612 extending from the base portion 610, a tip portion 614 and two slots 608 for aligning an insert 606 with reference to the cavity 604. Each of the portions 610, 612, 614 defining a negative shape of the corresponding portions of the needle to be fabricated. The insert 606 is in the form of an elongate pin structure comprising a plurality of integral rectangular sections 606(a)-(c) with different widths/diameters generally corresponding to the size of the corresponding lumen sections of the needle to be fabricated. The diameter of the sections 606(a)-(c) of the insert 606 are in descending size starting from the base section 606(a).

During fabrication of the needle, the pin structure 606 is inserted into the mould halves 600 and the pin structure 606 is aligned with the cavity 604 such that a leading edge 620 of the pin 606, in this case, leading corners 622 of the rectangular section 606(c) of the pin 606 intersect with the periphery of the cavity 604 at two locations (i.e. at two opposing walls defining the cavity) to form, the side ports (e.g. the side ports 108 of the needle 100 in FIG. 1(a)). The dashed lines represent the position of the pin in the mould cavity 604 when the pin 606 is inserted into the mould halve 600. Accordingly, the two slots 608 are shaped to align and hold the pin 606 in position within the mould halve 600. The shape of the slots 608 corresponds to the shape of the leading corners 612 of the pin 606 to align and hold the pin 606 within the mould halves 600.

The needles described above are fabricated as an integral piece and can be formed by, for example, injection moulding. Other types of moulding can also be used, for example, cast moulding, or derivatives of cast moulding or injection moulding such as thermoforming, insert moulding, compression moulding, transfer moulding, multi-shot moulding, etc. The derivatives can incorporate various add-on features to achieve specific process requirements. The add-on features may comprise external systems such as vacuum pump, robotic arms, etc. The various portions (i.e. the base portion, the stem portion and the tip portion) of the needles described in above can be made from the same material. It will be appreciated that the various portions of the needles can be made from different materials. For example, the stem portion and the tip portion of the needle can be made from a material with particular mechanical and bio-compatibility properties while the base portion is made from a different material with different mechanical and chemical properties. Different materials can be used to fabricate the various portions of the needle by multiple moulding of the various portions using various moulding processes known in the art.

A pair of mould halves 600 are aligned and held together tightly such that the cavity on one mould halve 600 faces the cavity on the other mould halve, before the pin 606 is inserted into the mould and aligned with respect to the mould cavity 604. The periphery of the negative shape of the needle is substantially parallel to an interface of the mould halves 600 when the mould halves 600 are held together. The two mould halves 600 are closed and held tightly together before the pin 606 is inserted into the mould and aligned with respect to the mould cavity 604. The mould cavity 604 is then filled with a polymer melt, for example, by using injection moulding, to form the needle. The polymer melt (not shown) is injected into the mould with an appropriate pressure to substantially fill the mould cavity 604. The molten polymer will flow around the pin 606 such that portions of the mould cavity 604 occupied by the pin 606 are not filled with the polymer melt, thereby creating the lumen (e.g. lumen 118 of the needle 100 of FIG. 1(a)), while the intersection of the leading edge 620 of the pin 606 with the periphery of the mould cavity 604 creates the side ports of the needle (e.g. the side ports 108 of the needle 100 of FIG. 1(a)).

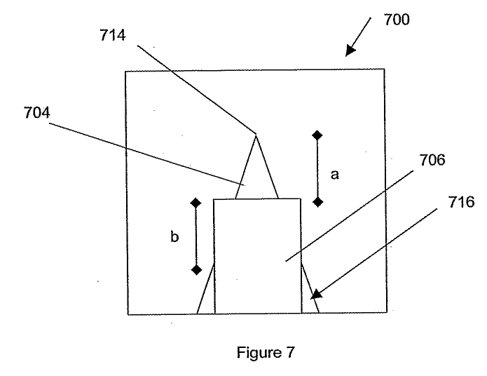

FIG. 7 is a schematic drawing of a portion of a mould halve 700 showing the tip portion 716 of the mould cavity 704. The mould halve 700 can, for example, be in the form of the mould halve 600 of FIG. 6. The submerge distance, a, of the needle (not shown in FIG. 7) is determined by the distance from a tip 714 of the cavity 704 to the intersection of the leading edge 710 of the pin 706 with the periphery of the cavity 704, while the extent to which the pin 706 intersects the periphery of the cavity 704 (i.e. distance b in FIG. 7) determines the size of the side ports in the needle to be fabricated.

It will be appreciated that the shape of the cavity in the mould halve depends on the shape of the needle to be fabricated and can be of various shapes other than triangular. If only one dispensing port is required in the needle, then the leading edge of the pin can be made to only intersect with the periphery of the cavity at one location. The number of side-ports created in the needle therefore depends on the number of locations where the leading edge of the pin intersects with the periphery of the mould cavity. This can be achieved by different combinations of the shape of the mould cavity and the shape of the pin. For example, a conical mould cavity with a hexagonal pin will have six ports, while a hexagonal pyramid cavity with a circular pin will also have six dispensing ports. The pin can be of other shapes, for example, cylindrical, polygonal, etc., instead of rectangular, depending on design requirements. Accordingly, depending on the geometry of the pin, the slot can be round or polygonal, etc.

It will be appreciated that the mould or mould halve 600 of FIG. 6, for example, be modified to comprise a plurality of cavities 604 arranged side by side. Furthermore, a plurality of moulds can, for example, be stacked onto one another to fabricate a plurality of needles.

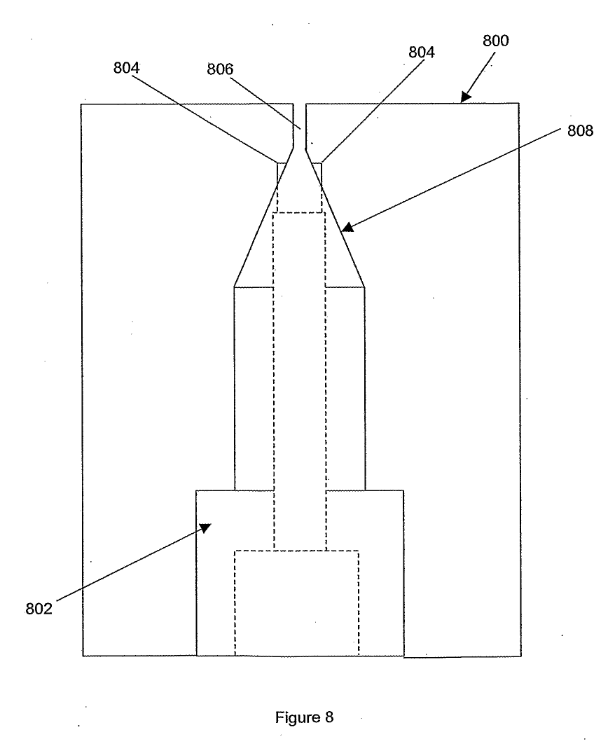

A schematic drawing of another mould halve 800 used for fabricating a needle is shown in FIG. 8. The mould halve 800 in FIG. 8 can be used to fabricate, for example, the needle 400 in FIG. 4. It will be appreciated that a mould (not shown in FIG. 8) comprising two identical mould halves 800 is used for fabricating the needle. Therefore, only one mould halve 800 is shown and described.

The mould cavity 802 comprises two slots 804 for aligning and holding a pin (not shown in FIG. 8), and vent 806 configured for creating the extended tip portion 424 of the needle 400 in FIG. 4. The vent 806 is formed on the tip portion 808 of the mould cavity 802. The vent 806 is generally an elongate channel extending at or near the tip portion 808 of the mould cavity 802.

The dashed lines in FIG. 8 show the position of the insert (not shown) when the insert is placed within the mould cavity 802.

The needle can be moulded by, for example, using injection moulding to fill the mould cavity 802 with polymer melt. A polymer melt (not shown) is injected into the mould with an appropriate pressure to substantially fill the mould cavity 802 in the mould.

Polymer melt is generally viscous and therefore has difficulty filling mould cavities. This becomes a significant problem when moulding objects of small dimensions, for example, at the tip portion of the needle where the dimensions can be about 10-100 microns in size. Air in the mould cavities is pushed by the polymer melt during moulding and trapped in the mould cavity forming voids in the moulded object. For example, air can be trapped near or at the tips of the mould cavities, causing the tip portion and the apex of in the resulting needle to be less sharp and not well defined.

By using a mould with a vent, for example, the vent 806 in FIG. 8, as the polymer melt fills the mould cavity 802, air trapped in the mould cavity 802 is vented out of the mould via the vent 806. When the mould cavity 802 is substantially filled with the polymer melt, additional pressure is applied to the polymer melt such that the polymer melt partially fills the vent 806 to form the extended tip portion of the needle to be fabricated, e.g. the extended tip portion 424 of the needle 400 in FIG. 4. Therefore, the vent 806 is configured for creating the extended tip portion 424 of the needle 400 in addition to provide for venting of air out of the mould cavity 802. The length of the extended tip portion 424 of the needle 400 is accordingly determined by the level of polymer melt filling the vent 806. Injection moulding can be carried out in a vacuum chamber to remove any residual air trapped in the mould cavities and to assist the polymer melt in entering and filling the needle cavities. Further, a vacuum oven can be used to melt the polymer.

It will be appreciated that the mould halve 600 shown in FIG. 6 can also comprise a vent formed on the tip portion 614 of the mould cavity 604 as described above for venting of air out of the mould cavity 604. In order to create needles without the extended tip, for example, the needle 100 in FIGS. 1(a) and 1(b), an appropriate pressure is applied to the polymer melt during the injection molding process such that the polymer melt does not fill the vent.

It will be appreciated that the pin can have different geometries and sections with different widths/diameters, depending on design requirements. The shape of lumen created will depend on the geometry of the pin. In general, the lumen defines a periphery of a negative shape of the pin. The pin may comprise different geometry and/or dimension at a stem portion and at the leading edge, for example, when a larger lumen is required to reduce dispensing pressure of the drug and a particular size of side-ports are required. Alternatively, the pin can be made tapered.

As described above, the mould halves (e.g. the mould halve 800 in FIG. 8 or the mould halve 600 FIG. 6) used to fabricate the needles comprises cavities that generally define a periphery of a negative shape of the needles to be fabricated. One way of creating the mould halve is to first create a master mould comprising a positive shape of the needles. In other words, the master mould comprises protrusions that are generally corresponding to the shape of the needles that are to be fabricated. The protrusions on the master mould can be fabricated by precision wire-cutting or other precision engineering means. The pattern of the protrusions corresponding to the shape of the needles on the master mould is then transferred onto a production mould or an intermediate mould by, for example, hot embossing, to form cavities corresponding to a negative shape of the needles.

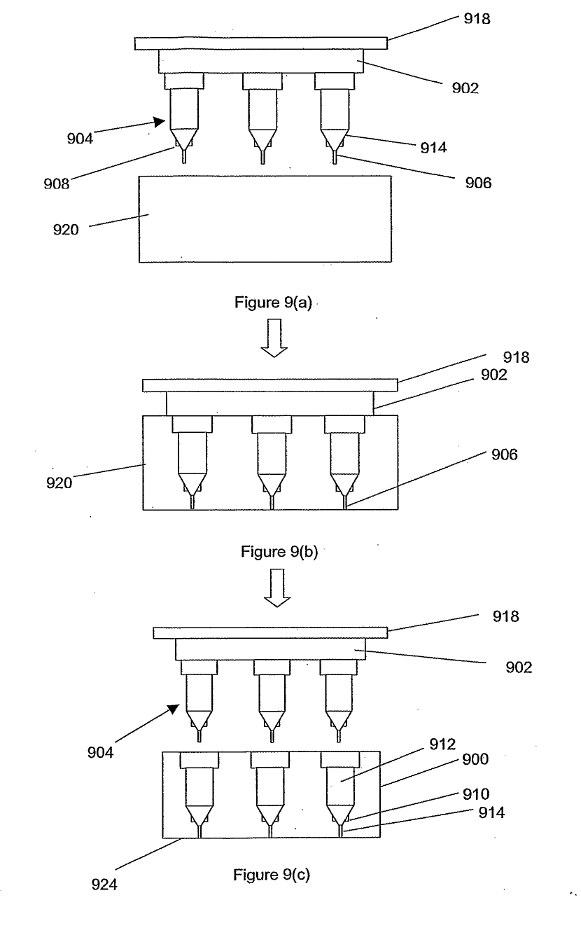

Schematic drawings showing a top view of a process for making a production mould halve 900 (FIG. 9(c)) for use in fabricating needles are shown in FIGS. 9(a)-9(c). The production mould halve 900, can be in the form of, for example, the mould halve 800 in FIG. 8, and can be used to form, for example, the needle 400 of FIG. 4. The master mould 902 comprises a plurality of protrusions 904 corresponding to the shape of the needles (e.g. needle 400 of FIG. 4) to be fabricated is first created. The master mould 902 is made of tool steel. The protrusions 904 on the master mould 902 can be made by precision wire cutting. Generally, the shape of the protrusions 904 on the master mould 902 are positive images of the needles to be fabricated. The protrusions 904 comprise extended tip portions 906. The extended tip portions 906 of the protrusions 904 are used to form vents 914 in the production mould halve 900. Each protrusion 904 comprises two triangular shaped shoulder portions 908 protruding from two opposing sidewalls 914. The two triangular shaped shoulder portions 908 are used to form the slots 910 in the cavity 912 of the production mould halve 900. It will be appreciated that the shoulder portions 916 can be of other geometries depending on the shape of the slot 910 that is required. Generally, a shape of the shoulder portions 916 is a positive shape of the slots 910 in the production mould halve 900. In this case, since the pin (not shown) is rectangular in shape, the shape of the slots 910 are triangular in shape and therefore, the shoulder portions 916 are also triangular in shape.

The master mould 902 mounted to a first heated platen 918 is hot embossed into a metal substrate 920 mounted on a second heated platen (not shown) to form a negative image of the master mould 902 (and therefore a negative image of the needles) in the substrate 920. The metal substrate 920 is placed below the master mould 902 and the master mould 902 is lowered onto the metal substrate 920 to transfer the shape of the needles on the master mould 902 onto the metal substrate 920 to form the cavities 912 comprising a periphery of a negative shape of the needles on the metal substrate 920. The master mould 902 is then lifted from the metal substrate 920 after embossing (FIG. 9(c)). The embossed substrate 920 forms the production mould halve 900 that is used for fabricating the needles. Two identical mould halves 900 are closed together tightly to form a mould (not shown). An edge portion 924 of the production mould halve 900 is removed, for example, by grinding, such that the vents 914 extend through the production mould halve 900. It will be appreciated that alternatively, the extended tip portions 906 of the master mould 900 can be made longer such that the vents 914 extend through the edge portion 924 of the substrate 920 during hot embossing (i.e. no grinding of the edge portion 924 is required).

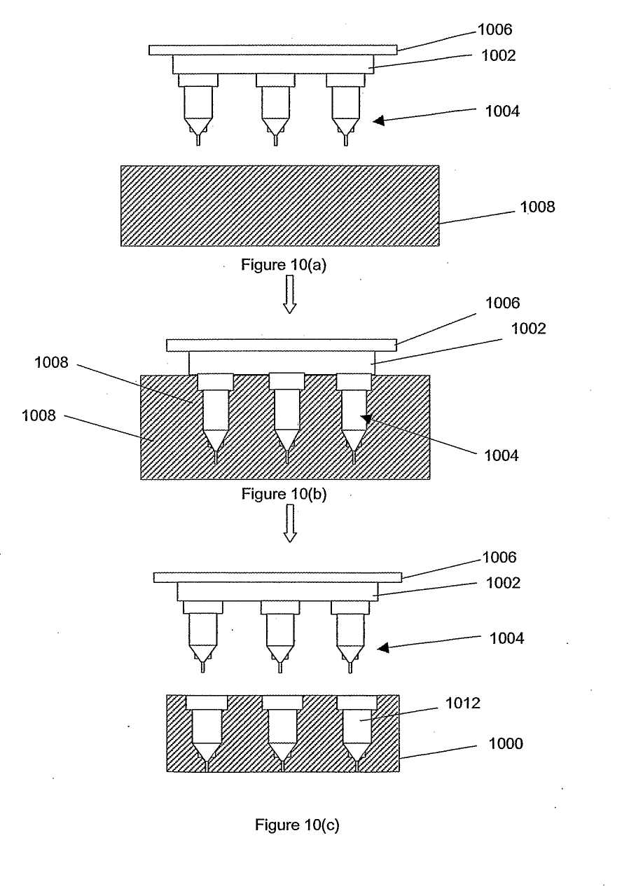

Schematic drawings showing a top view of another process for making a production mould halve 1000 for use in fabricating needles are shown in FIGS. 10(a)-10(c). The production mould halve 1000, can be in the form of, for example, the mould halve 800 in FIG. 8, and can be used to form, for example, the needle 400 of FIG. 4. In this example, microforming is used to fabricate the needles. The master mould 1002 comprising the protrusions 1004 is mounted to a punch 1006. A metal sheet 1008 is placed on a deformable die (not shown) such that the metal sheet 1008 is between the punch 1006 and the deformable die. The metal sheet 1008 can be made of metallic materials such as steel, aluminium and copper. The thickness of the metal sheet 1008 can range from about 0.05 mm to about 0.5 mm. However, other dimensions, for example, a thickness of greater than 0.5 mm can also be used depending on design requirements. The punch 1006 together with the master mould 1002 is moved towards the metal sheet 1008 and presses against the metal sheet 1008 and the deformable die. The metal sheet 1008 is deformed and a negative shape of the protrusions 1004 of the master mould 1002 is formed onto the metal sheet 1008. Therefore, the metal sheet 1008 comprises cavities 1012 generally defining the periphery of the negative shape of the needles to be formed. At the same time, the deformable die is also deformed by the protrusions 1004 of the master mould 1002 to form cavities defining the negative shape of the protrusions 1004. The punch 1004 together with the master mould 1002 is then retracted (FIG. 10(c)). The deformed metal sheet 1008 in FIG. 10(b) is removed and a thickness reinforcement process, for example, metal deposition is performed on an underside (not shown) of the metal sheet 1008 defining an exterior surface of the cavities 1012 to form the production mould halve 1000 used for fabricating the needles. Other methods of fabricating the production mould halve are also possible, for example, by first fabricating an intermediate mould halve using the master mould and subsequently fabricating the production mould halve using the intermediate mould halve.

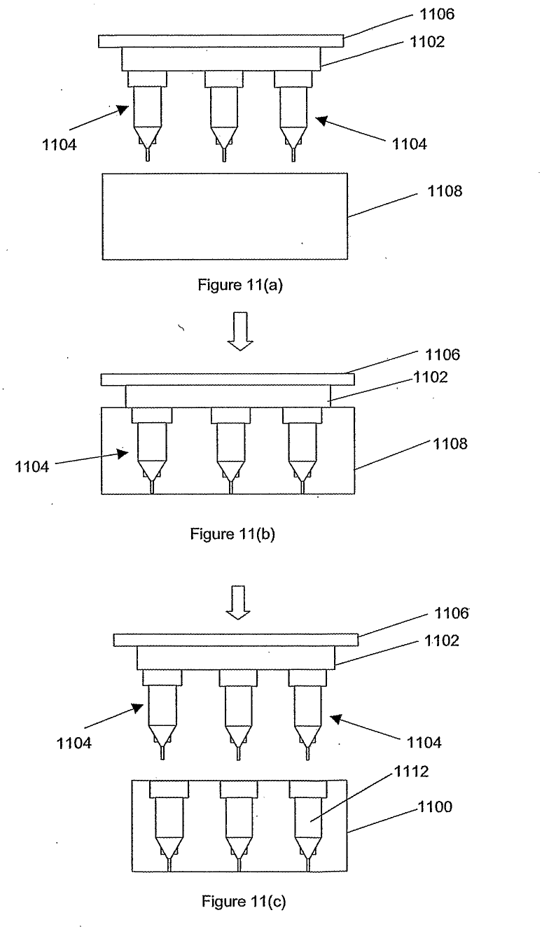

Schematic drawings showing a top view of a process for making an intermediate mould halve 1100 are shown in FIGS. 11(a)-11(c). As described earlier, the master mould 1102 comprises protrusions 1104 that are generally corresponding to the shape of the needles that are to be fabricated. The protrusions 1104 on the master mould 1104 are made of tool steel and can be fabricated by precision wire-cutting or other precision engineering means. The master mould 1102 mounted to a first heated platen 1106 is hot embossed into a polymer substrate 1108 mounted on a second heated platen (not shown) to form a negative image of the protrusions 1104 of the master mould 1102 (and therefore a negative image of the needles) in the polymer substrate 1108. The resulting embossed polymer substrate 1100 (i.e. the intermediate mould) comprising cavities 1112 defining a negative image of the protrusions (and therefore, a negative image of the needles) is then used to fabricate the production mould halve (not shown in FIGS. 11(a) to 11(c)) for fabricating needles.

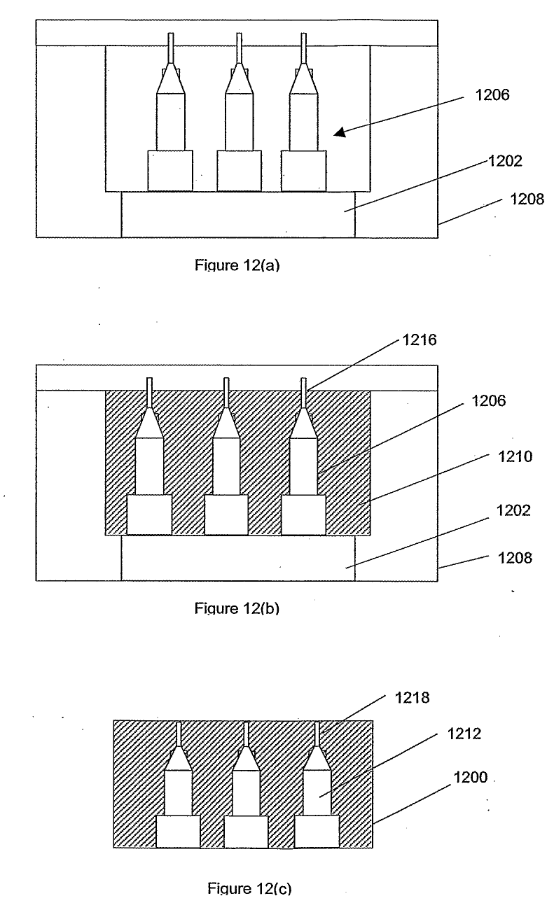

Schematic drawings showing a top view of another process for making an intermediate mould halve 1200 are shown in FIGS. 12(a)-12(c). In this example process, the intermediate mould halve 1200 is cast moulded. The master mould 1202 comprising the protrusions 1206 forms part of a casting mould 1208 used in the casting process. Molten polymer 1210 into the casting mould 1208 and over the master mould 1202 as shown in FIG. 12(b). The molten polymer 1210 is allowed to cure and removed from the casting mould 1208 to obtain the intermediate mould halve 1200 comprising cavities 1212 defining a negative shape of the needles to be fabricated. Each protrusion 1206 includes an extended tip portion 1216 for forming an air vent 1218 in the intermediate secondary polymeric mould 1200.

The polymeric intermediate mould halves described in FIGS. 11(a)-11(c) and FIGS. 12(a)-12(c) may be made of various types of polymers such as polycarbonate (PC), polystyrene (PS), polyetherimide (PEI), polypropylene (PP), polyethylene (PE), etc. in sheet form or poly(dimethylsiloxane) (PDMS) in liquid form for cast moulding.

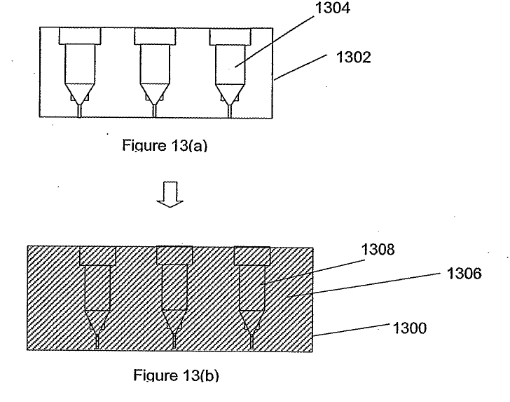

Schematic drawings showing a top view of a process for making a production mould halve 1300 using an intermediate mould halve 1302 are shown in FIGS. 13(a) and 13(b). The intermediate mould halve 1302 can, for example, be in the form of the intermediate mould halve 1200 in FIGS. 12(a)-12(c) or the intermediate mould halve 1100 in FIGS. 11(a)-11(c). As described above, the intermediate mould halve 1302 comprises cavities 1304 defining a negative shape of the needles to be fabricated. A layer of metal 1306 (represented by the shaded regions in FIG. 13(b)) is deposited onto the intermediate mould halve 1302 and into the cavities 1304 of the intermediate mould halve 1302 (e.g. by chemical or vapour deposition, electroforming, electroplating, etc.). The deposited layer of metal 1306 comprising cavities 1308 is removed from the intermediate mould halve 1302 as shown in FIG. 15(b). The deposited layer of metal 1306 is subjected to a thickness reinforcement process, for example, metal deposition performed on an underside (not shown) of the deposited metal layer 1306 defining an exterior surface of the cavities 1308 to form the production mould halve 1300 used for fabricating the needles.

In the embodiments described above, the polymer used for making the intermediate mould halve is chosen to have a certain elasticity and deformability characteristic such that the intermediate mould halves can be re-used. A mould release agent can be applied or sprayed onto the intermediate mould before metal deposition is performed on the intermediate mould. This allows effective release of the deposited metal layer and at the same time preserves the intermediate mould halve from damage to allow subsequent use.

Since the needles described above and the base portion in the form of e.g. a luer-lock connector are formed as an integral piece, separate assembling of the needle and the luer-lock connector is eliminated. Machining costs are also eliminated, as post fabrication machining is not required. On the other hand, conventional steel needles are required to be separately assembled to a luer-lock connector and may require post fabrication machining. Further, ploymeric needles have the freedom to take any other form which may be impossible or not cost effective for the fabrication of stainless steel needles.

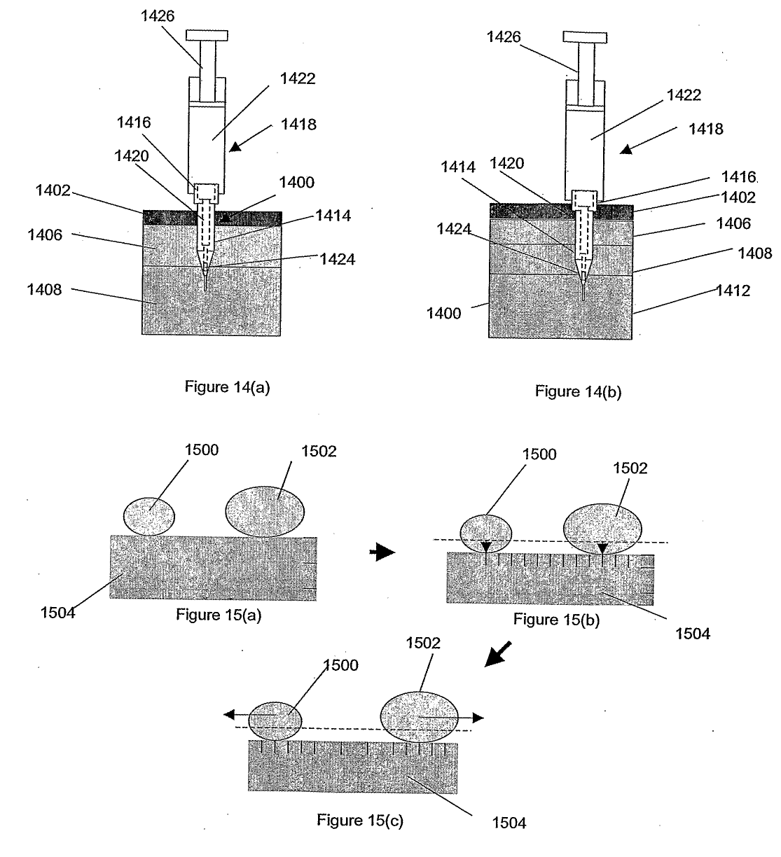

FIGS. 14(a) and 14(b) are schematic drawings illustrating intradermal, and subcutaneous drug delivery modes, respectively.

The intradermal delivery mode (see FIG. 14(a)) involves insertion of needle 1400 past the stratum corneum (SC) 1402 and into the epidermis 1406 and/or the dermis 1408 layer of the skin (i.e. within the thickness of the skin). Typically the SC 1402 is approximately 10-20 μm, the epidermis 1406 is approximately 0.1-0.2 mm (i.e. 100-200 μm) and the dermis 1414 is approximately 1.0-2.0 mm. The length of the stem portion 1414 of the needle 1400 is chosen such that the needle can be used for intradermal and subcutaneous drug delivery, for example, by injecting a liquid into the body. As described above, the base portion 1416 of the needle 1400 comprises a connection adapter (e.g. a luer-lock connector) for coupling to a dispensing apparatus such as a syringe 1418 such the lumen structure 1420 is in fluid communication with a reservoir 1422 within the syringe 1418. During drug delivery, drugs stored in the reservoir 1422 are dispensed into the dermis layer 1408 of the skin via the side ports 1424.

The needle 1400 can also be used for extracting body fluid, which may include whole blood sampling. Body fluid can be extracted from the body e.g. from blood capillaries, via the side ports 1424 and the lumen structure 1420 for whole blood sampling. The extracted body fluid can be stored in the reservoir 1422 in the syringe 1418.

Alternatively, the base portion 1416 can comprise a barrel portion adapted for coupling to a plunger 1426.

The base portion 1416 of the needle 1400 can be made from a different material from the stem portion 1414 and the tip portion 1424 of the needle 1400. Since the base portion 1416 is used for coupling to a dispensing apparatus, a material with suitable mechanical properties is used to make the base portion 1416. On the other hand, since the tip portion 1242 and the stem portion 1414 are to be inserted into the body, the tip portion 1242 and the stem portion 1414 can be made from a material with bio-compatible properties. It will be appreciated that the entire needle 1400 can also be made from a single material.

The subcutaneous delivery mode (see FIG. 14(b)) involves insertion of a needle 1400 beyond the thickness of the skin where the drug can be injected, for example, in between the skin and muscles 1412. Further drug delivery modes, for example, intravenous delivery (not shown) involving insertion of the needle into a vein for direct injection of drug into blood streams and intramuscular delivery (not shown) involving insertion of the needle into muscles are generally classified as a type of subcutaneous delivery mode as both the intravenous and intramuscular delivery modes involve insertion of the needle beyond the thickness of the skin.

The penetration depth of the needle is linearly related to the length of the needle (in particular the stem portion of the needle) when it is fully plunged into the skin. Although a significant portion of the needle's length goes into the skin after penetration, a portion of the needle, for example the base portion and a portion of the stem portion of the needle stays outside of but in contact with the skin due to skin deformation. For this reason, the length of the stem portion required should generally be greater than a targeted penetration depth. The extended tip portion of the needles (e.g. the needle 400 in FIG. 4) can reduce the discrepancy between the length of the needle and the targeted penetration depth as less deformation of the skin occurs, thereby achieving more accurate penetration depth.

One way to achieve variable penetration depth using a needle is to restrict the penetrating length of the stem portion of the needle by an external means, for example a mechanical limiter with a turn-able cap and screw thread, or any other similar mechanical means that provides similar functions, to adjust the penetrating length of the stem portion of the needle. Alternatively a double-sided adhesive tape with a desired thickness that provides a clearance distance can be disposed near or at the base portion of the needles to limit the depth of penetration of the stem portion of the needle. The adhesive tape may have an elastic characteristic such that the adhesive tape springs back to its original thickness after the initial compression during penetration. The adhesive tape also provides fixation of the needle module onto the skin after breaching of the SC. Another way is to stop the advancing stroke of the needle once the desired depth is reached.

The penetration depth of the needle can also be limited by covering the base portion or the stem portion near the base portion of the needle with a solid layer (rigid epoxy or compressible adhesive tape) thereby reducing the effective length of the stem portion of the needle.

Skin tensioning and compression may be required prior to the breaching of the SC for intradermal or subcutaneous drug delivery to increase penetration effectiveness. This can be achieved, for example, by using a middle 1500 finger and a thumb 1502 to compress the skin (FIG. 15(b)) and tension the skin 1504 (FIG. 15(c)), and pressing the skin with the needle platform using appropriate force, as shown in FIGS. 15(a) to 15(c).

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1. A needle comprising:
a base portion;
a stem portion extending from the base portion;
a tip portion formed on the stem portion, the tip portion having a solid apex;
a lumen extending within the needle, the lumen extending from the base portion to the stem portion and the tip portion;
wherein a length of the stem portion is chosen such that the needle can be used for intradermal or subcutaneous drug delivery or body fluid extraction, and the base portion, the stem portion and the tip portion are integrally formed.

2. A needle as claimed in claim 1, wherein the base portion, the stem portion and the tip portion are integrally formed from the same material.

3. The needle as claimed in claim 1, wherein at least one of the base portion, the stem portion or the tip portion is integrally formed from a different material.

4. The needle as claimed in claim 1, further comprising:
at least one side port, wherein the side port extends into the lumen such that the side port and the lumen are in fluid communication with each other.

5. The needle as claimed in claim 4, wherein the lumen comprises a plurality of sections, each section having a different diameter or width.

6. The needle as claimed in claim 5, wherein the diameter or widths of the plurality of sections is in descending order starting from a base section of the lumen.

7. The needle as claimed in claim 4, wherein the lumen is tapered.

8. The needle as claimed in claim 3, comprising: two or more side ports disposed on the tip portion of the needle.

9. The needle as claimed in claim 1, further comprising an extended tip portion formed on the tip portion, the extended tip portion forming an apex of the needle, wherein the extended tip portion comprises a substantially elongate protrusion extending from the tip portion.

10. The needle as claimed in claim 9, wherein a geometry of the extended tip portion is substantially the same as a geometry of the tip portion.

11. The needle as claimed in claim 1, wherein the base portion comprises a connection adapter for coupling to a dispensing apparatus.

12. The needle as claimed in claim 1, wherein the base portion comprises a luer lock structure for interconnecting the needle to a syringe.

13. The needle as claimed in claim 1, wherein the base portion comprises a barrel portion, the barrel portion being adapted for coupling to a plunger.

14. The needle as claimed in claim 1, wherein the needle is made from at least one of a group of polymers consisting of polycarbonate (PC), polystyrene (PS), polyetherimide (PEI), or polyetherehterketone (PEEK).

15. The needle as claimed in claim 1, wherein the needle is fabricated using injection moulding or cast moulding or derivatives of cast moulding or injection moulding.

16. A method of fabricating a needle, the method comprising:
forming a base portion;
forming a stem portion extending from the base portion;
forming a tip portion formed on the stem portion, the tip portion having a solid apex;
forming a lumen extending within the needle, the lumen extending from the base portion to the stem portion and the tip portion;
wherein a length of the stem portion is chosen such that the needle can be used for intradermal or subcutaneous drug delivery or body fluid extraction, and the base portion, the stem portion and the tip portion are integrally formed.

17. The method as claimed in claim 16, wherein the base portion, the stem portion and the tip portion are integrally formed from the same material.

18. The method as claimed in claim 16, wherein at least one of the base portion, the stem portion or the tip portion is integrally formed from a different material.

19. The method as claimed in claim 16, further comprising:
forming at least one side port, wherein the side port extends into the lumen such that the side port and the lumen are in fluid communication with each other.

20. The method as claimed in claim 16, wherein the lumen comprises a plurality of sections, each section having a different diameter or width.

21. The method as claimed in claim 20, wherein the diameter or widths of the plurality of sections is in descending order starting from a base section of the lumen.

22. The method as claimed in claim 19, wherein the lumen is tapered.

23. The method as claimed in claim 19, the method further comprising: two or more side ports disposed on the tip portion of the needle.

24. The method as claimed in claim 16, further comprising: forming an extended tip portion on the tip portion, wherein the extended tip portion forms an apex of the needle and the extended tip portion comprises a substantially elongate protrusion extending from the tip portion.

25. The method as claimed in claim 16, wherein the base portion comprises a connection adapter for coupling to a dispensing apparatus.

26. The method as claimed in claim 16, wherein the base portion comprises a luer lock structure for interconnecting the needle to a syringe.

27. The method as claimed in claim 16, wherein the base portion comprises a barrel portion, the barrel portion being adapted for coupling to a plunger.

28. The method as claimed in claim 16, further comprising:
forming a master mould comprising a positive shape of the needle;
using the master mould to form a production mould comprising a cavity comprising a periphery of a negative shape of the needle; and
using the production mould to form the needle.

29. The method as claimed in claim 28, comprising:
forming the master mould comprising a positive shape of the needle;
using the master mould to form an intermediate mould comprising a cavity comprising a periphery of a negative shape of the needle; and
using the intermediate mould to form the production mould.

30. The method as claimed in claim 28, comprising:
forming a pair of production mould halves, each production mould half comprising a cavity comprising a periphery of a negative shape of the needle;
using the pair of production mould halves to form the needle.

31. The method as claimed in claim 29, comprising:
forming a pair of intermediate mould halves, each intermediate mould half comprising a cavity comprising a periphery of a negative shape of the needle;
using the pair of intermediate mould halves to form a pair of production mould halves.

32. The method as claimed in claim 30, wherein the pair of production mould halves are identical to each other, each production mould half comprising a plurality of cavities, each cavity comprising a periphery of a negative shape of the needle.

33. The method as claimed in claim 30, wherein the periphery of the negative shape of the needle is substantially parallel to an interface of the two production mould halves.

34. The method as claimed in claim 30, wherein the intermediate mould halves are made from polymer.

35. The method as claimed in claim 30, further comprising: filling the cavity of the production mould halves with a polymer to form the needle.

36. The method as claimed in claim 28, wherein the positive shape of the needle on the master mould is created by precision wire-cutting.

37. The method as claimed in claim 30, wherein the production mould halves are made of metal.

38. The method as claimed in claim 30, the method further comprising:
providing an insert member;
providing the pair of production mould halves, each of the production mould halves further comprising at least one slot portion for receiving and aligning the insert member such that a leading edge of the insert intersects the periphery of the negative shape of the needle;
aligning the pair of production mould halves;
inserting the insert member into the production mould halves; and
filling the production mould halves with a fill material, wherein
the insert creates the lumen in the needle and the intersection of the leading edge of the insert with the periphery of the negative shape of the needle creates the side port extending into the lumen such that the lumen and the side port are in fluid communication with each other.

39. The method as claimed in claim 30, wherein the periphery of the negative shape of the needle comprises a channel extending from an apex of the negative shape of the needle for forming the extended tip portion of the needle.

40. The method as claimed in claim 39, further comprising filling the channel at least partially to form the extended tip portion.

41. The method as claimed in claim 38, wherein the insert member comprises a pin having a plurality of sections, each section having a different diameter or width.

42. The method as claimed in claim 41, wherein the pin is tapered.

43. A method of using a needle as claimed in claim 1 for injecting liquid into a body.

44. A method of using a needle as claimed in claim 1 for extracting body fluid from a body.

45. The method of using the needle as claimed in claim 44, wherein extracting body fluid from the body includes whole blood sampling.

46. The needle as claimed in claim 9, wherein a geometry of the extended tip portion is different from a geometry of the tip portion.
